(19) United States
(12) Patent Application Publication
(10) Pub. No.: US 2022/0162558 A1
(43) Pub. Date: May 26, 2022

(54) METHOD FOR INDUCING DIFFERENTIATION OF STEM CELLS INTO CHONDROCYTES BY USING OLIGOPEPTIDES

(71) Applicant: POSTERA HEALTH SCIENCE Inc., Hanam-si (KR)

(72) Inventors: Byeongmoon JEONG, Seoul (KR); Ye-Lin KIM, Namyangju-si (KR); Soyoun UM, Incheon (KR)

(21) Appl. No.: 17/600,940

(22) PCT Filed: Jan. 6, 2020
(86) PCT No.: PCT/KR2020/000193

§ 371 Date: Oct. 1, 2021

(30) Foreign Application Priority Data
Apr. 3, 2019 (KR) 10-2019-0039147

(51) Int. Cl. C12N 5/077; A61K 35/32; A61L 27/52; A61L 27/38; A61P 19/04

(52) U.S. Cl. CPC C12N 5/0655; A61K 35/32; A61L 27/52; C12N 2506/1346; A61P 19/04; C12N 2501/998; A61L 27/3817

(57) ABSTRACT

The present application relates to a method of inducing differentiation of stem cells into chondrocytes using an oligopeptide, and a pharmaceutical composition for treating cartilage injury disease containing differentiated chondrocytes obtained by the method.

### TECHNICAL FIELD

The present application relates to a method of inducing differentiation of stem cells into chondrocytes using an oligopeptide, and a pharmaceutical composition for treating cartilage injury disease containing differentiated chondrocytes obtained by the method.

### BACKGROUND ART

Stem cells are capable of self-renewal and have higher proliferative and regenerative potential than mature cells. In particular, for treatment for age-related degenerative diseases such as osteoarthritis, stem cells are expected to be superior to mature cells due to their rapid differentiation into chondrocytes and their differentiation into a chondrogenic lineage.

Stem cells, including mesenchymal stem cells (MSCs), also have the property of homing to inflammatory sites, and exhibit immunosuppressive and anti-inflammatory effects by inhibiting the proliferation of immune cells and the release of pro-inflammatory cytokines. Currently, cell therapy for damaged cartilage is performed using autologous chondrocytes and mesenchymal stem cells in hospitals. Autologous chondrocyte transplantation mainly leads to fibrocartilage tissue, which lacks mechanical properties as cartilage tissue and is degraded within 2 years.

Even in the case of stem cell therapy, stem cells do not differentiate into chondrocytes to become cartilage tissue, but rather secretions from stem cells mainly contribute to clinical efficacy. Various approaches have been tried to enhance chondrogenic differentiation of mesenchymal stem cells. These include: 1) protein or non-protein growth factors; 2) scaffold designs with different components, charges, hardness, and porosity; 3) co-culture with chondrocytes; 4) genetic modulation of stem cells; and 5) chemical stimuli such as oxygen partial pressure, electrical stimulation and mechanical stimulation. Among them, an approach employing a specific factor that induces chondrogenic differentiation of stem cells is considered the most promising method. Many factors have been developed to promote chondrogenic differentiation of mesenchymal stem cells. These factors include proteins (such as tissue growth factor-β1-3 (TGF-β1-3), fibroblast growth factor-2 (FGF-2), epithelial growth factor (EGF), insulin-like growth factor-1 (IGF-1), bone morphogenetic protein-2,4,7 (BMP-2,4,7), platelet-derived growth factor (PDGF), and interleukin-1β (IL-1β)) and small molecules (such as ethanol, prostaglandin E2, ascorbic acid, staurosporine, dexamethasone, and 1,25-dihydroxy vitamin D). Compared with protein-based growth factors, small molecular compounds are chemically well-defined, and thus are more reliable as a promoter of chondrogenic differentiation of mesenchymal stem cells. In addition, TGF-β, which is the most effective protein for promoting chondrogenic differentiation of mesenchymal stem cells, induces synovial fibrosis, osteophyte formation, and endochondral ossification. FGF-2 also induces histopathological chondrocyte clonal cluster formation, antagonizes proteoglycan synthesis, and increases an inflammatory marker of matrix metalloproteinase (MMP), and thus accelerates osteoarthritis development. Recently, it has been reported that kartogenin (KGN) and 5{I,2}, a derivative of oxopiperazine, are superior to TGF-β in promoting chondrogenic differentiation of mesenchymal stem cells, even though they are small molecular compounds.

Korean Patent Application Publication No. 10-2010-0069376 shows that adipose-derived mesenchymal stem cells are differentiated into chondrocytes by culturing these stem cells in a chondrogenic medium containing TGF-β2 or a combination of TGF-β2 and IGF-1.

It has been reported that Cartistem, a stem cell-based treatment developed by Medipost in 2012, is clinically effective due to the paracrine effects of stem cells rather than differentiated chondrocytes. In the process of finding molecules for inducing specific differentiation of mesenchymal stem cells, it has been suggested that small molecular compounds rather than macromolecular proteins or genetic modulation are clinically promising due to their superior and controllable biochemical advantages for reproducible production and rapid and reversible efficacy.

It has been reported that small molecular compounds including ethanol, ascorbic acid, dexamethasone, prostaglandin E2, staurosporin, catogenin, and oxopiperazine derivatives (5{I,2}) effectively induce chondrogenic differentiation of mesenchymal stem cells. However, effective monomolecular compounds for better chondrogenic differentiation have been continuously studied.

U.S. Pat. No. 9,487,754 B2 shows that differentiation of mesenchymal stem cells into chondrocytes is promoted by controlling the release of CTGF and TGFB3 from microspheres containing CTGF and TGFB3.

U.S. Pat. No. 9,464,065 B2 and Science, 2012, 336, page 717-721 show that differentiation of stem cells into chondrocytes is promoted using kartogenin and a derivative thereof.

Korean Patent No. 10-2013-0126018 and Chemical Science, 2012, 3, page 3071-3075 show that differentiation of stem cells into chondrocytes is promoted using the oxopiperazine derivative 5{I,2} and a derivative thereof.

### DISCLOSURE

### Technical Problem

The present application is intended to provide a method of inducing differentiation of stem cells into chondrocytes using an oligopeptide, and a pharmaceutical composition for treating cartilage injury disease containing differentiated chondrocytes obtained by the method.

However, the problem to be solved by the present application is not limited to the above-mentioned problem, and other problems not mentioned herein will be clearly understood by those skilled in the art from the following description.

### Technical Solution

A first aspect of the present application provides a composition for inducing differentiation of stem cells into chondrocytes, the composition containing an oligopeptide consisting of 2 to 10 amino acids.

A second aspect of the present application provides a method for inducing differentiation of stem cells into chondrocytes, the method including inducing differentiation into chondrocytes by treating the stem cells with an oligopeptide consisting of 2 to 10 amino acids.

A third aspect of the present application provides a pharmaceutical composition for treating cartilage injury disease, the pharmaceutical composition containing differentiated chondrocytes obtained by the method according to the second aspect of the present application.

A fourth aspect of the present application provides a hydrogel for inducing differentiation of stem cells into chondrocytes, the hydrogel containing the composition for inducing differentiation according to the first aspect of the present application.

### Advantageous Effects

According to embodiments of the present application, the use of an oligopeptide consisting of 2 to 10 amino acids, specifically 3 to 5 amino acids, may selectively induce the differentiation of stem cells into chondrocytes by enhancing chondrogenic differentiation of tonsil-derived mesenchymal stem cells while inhibiting osteogenic differentiation thereof. In addition, differentiated chondrocytes obtained by the induction method may be applied as a material for cartilage disease treatment or tissue engineering.

### BRIEF DESCRIPTION OF DRAWINGS

FIG. 1(a), FIG. 1(b), FIG. 1(c), FIG. 1(d) and FIG. 1(e) are graphs showing the mRNA expression levels of COL II (a), COMP (b), ACAN (c), COL X (d) and COL I α1 (e), analyzed by real time RT-PCR, in stem cells subjected to 3-dimensional pellet culture (n=3), according to one example of the present application.

FIG. 2 shows photographs obtained by three dimensionally culturing stem cells with each oligopeptide for 21 days, sectioning the cell pellets to a thickness of 7 μm, and then subjecting the sectioned pellets to alcian blue staining and safranin O staining (scale bar=0.5 mm), according to one example of the present application.

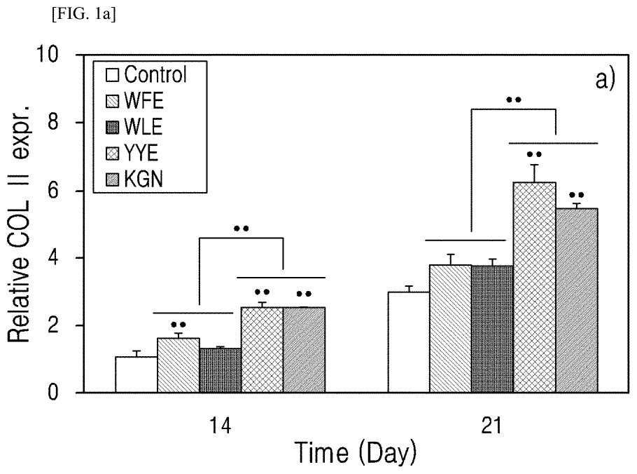

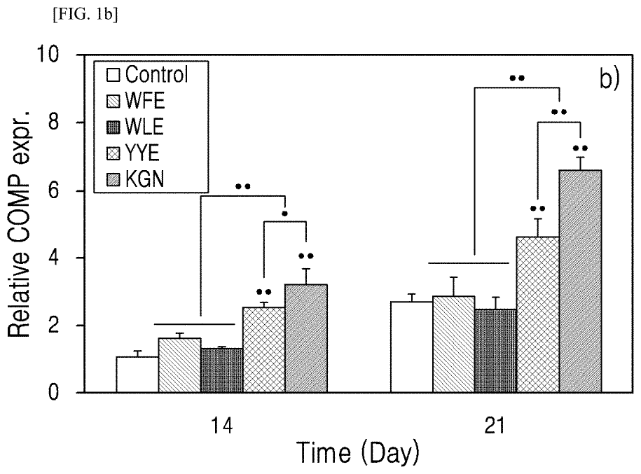

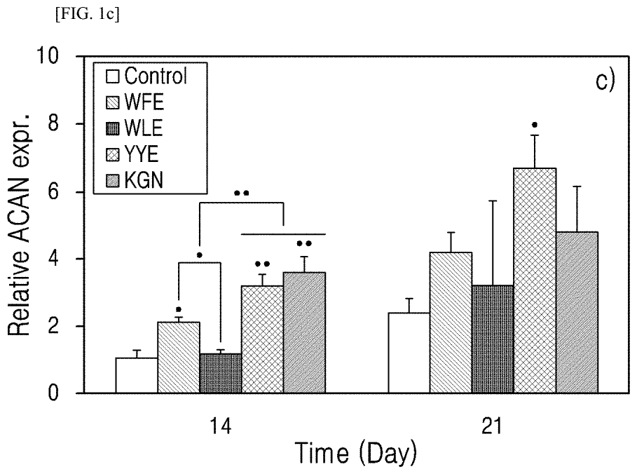

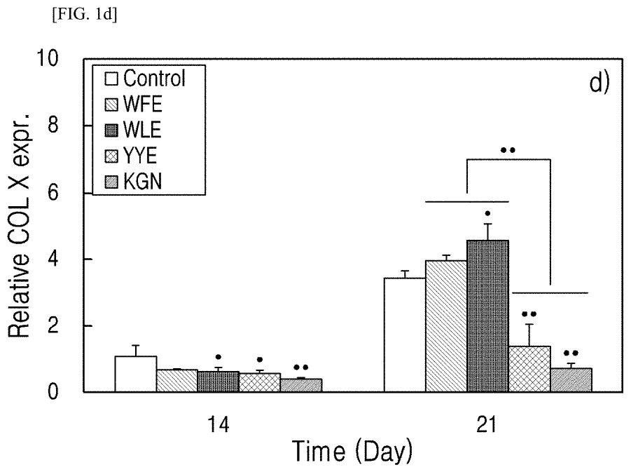

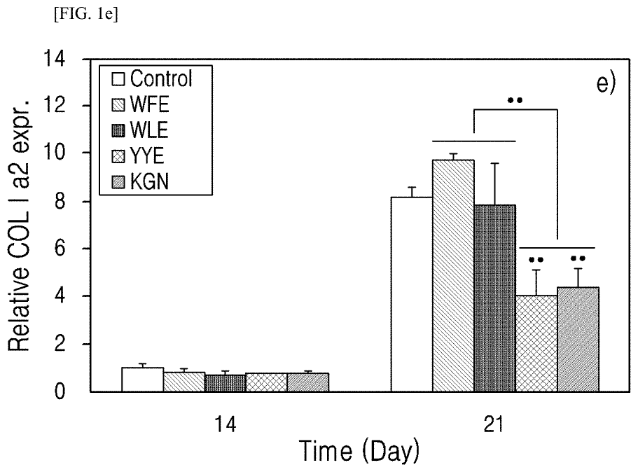

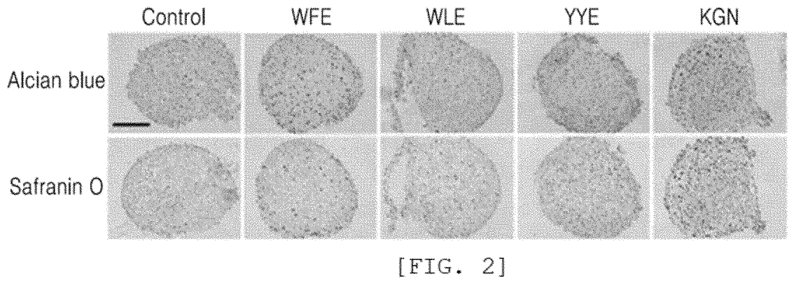

### BEST MODE

Hereinafter, embodiments of the present application will be described in detail with reference to the accompanying drawings so that the present application can be easily carried out by those of ordinary skill in the art to which the present application pertains. However, the present application may be embodied in various different forms and is not limited to the embodiments described herein. In the drawings, parts irrelevant to the description are omitted in order to clearly explain the present application, and like reference numerals denote like parts throughout the specification.

Throughout the present specification, it is to be understood that, when any part is referred to as being “connected” to another element, it not only refers to a case where any part is connected to the other part but also a case where intervening elements may be present therebetween.

Throughout the present specification, when any member is referred to as being “on” another member, it not only refers to a case where any member is in contact with another member, but also a case where a third member exists between the two members.

Throughout the present specification, it is to be understood that when any part is referred to as “including” any component, it does not exclude other components, but may further include other components, unless otherwise specified. Throughout the specification, words of degree, such as “about”, “substantially”, and the like are used herein in the sense of “at, or nearly at, when given the manufacturing and material tolerances inherent in the stated circumstances” and are used to prevent the unscrupulous infringer from unfairly taking the stated disclosure where exact or absolute figures are stated as an aid to understanding the present application. As used throughout the present specification, the term “step of doing . . . ” or “step of . . . ” does not mean “step for doing . . . .”

Throughout the present specification, the term “combination(s) thereof” included in the expression of the Markush form means one or more mixtures or combinations selected from the group consisting of the components described in the Markush form representation, and includes one or more selected from the group consisting of the above components.

Throughout the present specification, the expression “A and/or B” means “A or B” or “A and B”.

Hereinafter, embodiments and examples of the present application will be described in detail with reference to the accompanying drawings. However, the present application may not be limited to these embodiments and examples and the accompanying drawings.

Throughout the present specification, “differentiation” refers to a phenomenon in which cells becomes more specialized in structure or function during growth after division and proliferation thereof, that is, cells, tissues, etc. of an organism change in their shape or function to perform the given task.

Throughout the present specification, the term “chondrocytes” may include chondrocytes that have differentiated from stem cells, or cells undergoing differentiation into chondrocytes.

Throughout the present specification, the term “medium” refers to a mixture for in vitro culture or differentiation of cells such as stem cells, which contains components essential for growth and proliferation of the cells, such as sugar, amino acids, various nutrients, serum, growth factors, minerals, and the like.

A first aspect of the present application provides a composition for inducing differentiation of stem cells into chondrocytes, the composition containing an oligopeptide consisting of 2 to 10 amino acids.

In one embodiment of the present application, the expression of genes or proteins contained in the chondrocytes differentiated from stem cells may be amplified by the composition for inducing differentiation containing the oligopeptide. For example, the genes or proteins may include biomarkers. Specifically, the genes or proteins may be selected from the group consisting of collagen type II alpha 1 (COL II), SRY-box 9 (SOX 9), cartilage oligomer matrix protein (COMP), agrican (ACAN), collagen type X alpha 1 (COL X), collagen type I alpha 2 (COL I α2), glyceraldehyde 3-phosphate dehydrogenase (GAPDH), sulfated glycosaminoglycan (sGAG), proteoglycans, and combinations thereof, but are not limited thereto.

In one embodiment of the present application, the oligopeptide may comprise, but is not limited to, 2 to 10 amino acids selected from the group consisting of glycine, alanine, valine, leucine, isoleucine, phenylalanine, proline, tyrosine, tryptophan, cystine, methionine, serine, threonine, lysine, arginine, histidine, aspartic acid, glutamic acid, asparagine, glutamine, and combinations thereof.

In one embodiment of the present application, the oligopeptide may consist of 2 to 10, 2 to 8, 2 to 6, or 3 to 5 amino acids.

In one embodiment of the present application, the oligopeptide may consist of, but not limited to, an amino acid sequence of three amino acids, for example, alanine-alanine-glutamic acid, phenylalanine-phenylalanine-glutamic acid, isoleucine-phenylalanine-glutamic acid, leucine-phenylalanine-glutamic acid, methionine-phenylalanine-glutamic acid, valine-phenylalanine-glutamic acid, tryptophan-phenylalanine-glutamic acid, tyrosine-phenylalanine-glutamic acid, phenylalanine-arginine-aspartic acid, phenylalanine-leucine-glutamic acid, tryptophan-leucine-glutamic acid, tyrosine-isoleucine-aspartic acid, tyrosine-tyrosine-aspartic acid, or tyrosine-tyrosine-glutamic acid.

In one embodiment of the present application, the oligopeptide may consist of, but not limited to, an amino acid sequence of tryptophan-phenylalanine-glutamic acid, tryptophan-leucine-glutamic acid, or tyrosine-tyrosine-glutamic acid.

In one embodiment of the present application, the degree of differentiation of stem cells into chondrocytes may be different depending on, but not limited to, the amino acid sequence of the oligopeptide.

In one embodiment of the present application, the concentration of the oligopeptide may be in the range of, but not limited to, about 10 nM to about 100 μM.

In one embodiment of the present application, if the concentration of the oligopeptide is lower than about 0.01 μM or higher than about 100 μM, the effect of the oligopeptide on differentiation into chondrocytes may be lowered. Thus, the concentration of the oligopeptide is preferably in the range of about 0.01 μM to about 100 μM, for example, about 0.01 μM to about 100 μM, about 0.01 μM to about 75 μM, about 0.01 μM to about 50 μM, about 0.01 μM to about 10 μM, about 0.01 μM to about 1 μM, about 0.01 μM to about 0.1 μM, about 0.1 μM to about 100 μM, about 0.1 μM to about 75 μM, about 0.1 μM to about 50 μM, about 0.1 μM to about 10 μM, about 0.1 μM to about 1 μM, about 1 μM to about 100 μM, about 1 μM to about 75 μM, about 1 μM to about 50 μM, about 1 μM to about 10 μM, about 10 μM to about 100 μM, about 10 μM to about 75 μM, about 10 μM to about 50 μM, about 50 μM to about 100 μM, about 50 μM to about 75 μM, or about 75 μM to 100 μM.

In one embodiment of the present application, the stem cells may include, but are not limited to, those selected from the group consisting of mesenchymal stem cells, induced pluripotent stem cells, embryonic stem cells, and combinations thereof.

In one embodiment of the present application, the mesenchymal stem cells may include, but are not limited to, those selected from the group consisting of bone marrow-derived stem cells, adipose-derived stem cells, tonsil-derived stem cells, synovial stem cells, and combinations thereof.

In one embodiment of the present application, the mesenchymal stem cells may be obtained from, but not limited to, bone marrow, tissue, embryo, umbilical cord blood, blood, or body fluid.

A second aspect of the present application provides a method for inducing differentiation of stem cells into chondrocytes, the method including inducing differentiation into chondrocytes by treating the stem cells with an oligopeptide consisting of 2 to 10 amino acids.

With respect to the method for inducing differentiation according to the second aspect of the present application, detailed description of parts overlapping with those in the first aspect of the present application are omitted, but the contents described in the first aspect of the present application may be equally applied to the second aspect.

In one embodiment of the present application, the oligopeptide may include, but is not limited to, amino acids selected from the group consisting of glycine, alanine, valine, leucine, isoleucine, phenylalanine, proline, tyrosine, tryptophan, cystine, methionine, serine, threonine, lysine, arginine, histidine, aspartic acid, glutamic acid, asparagine, glutamine, and combinations thereof.

In one embodiment of the present application, the oligopeptide may consist of, but not limited to, an amino acid sequence of alanine-alanine-glutamic acid, phenylalanine-phenylalanine-glutamic acid, isoleucine-phenylalanine-glutamic acid, leucine-phenylalanine-glutamic acid, methionine-phenylalanine-glutamic acid, valine-phenylalanine-glutamic acid, tryptophan-phenylalanine-glutamic acid, tyrosine-phenylalanine-glutamic acid, phenylalanine-arginine-aspartic acid, phenylalanine-leucine-glutamic acid, tryptophan-leucine-glutamic acid, tyrosine-isoleucine-aspartic acid, tyrosine-tyrosine-aspartic acid, or tyrosine-tyrosine-glutamic acid.

In one embodiment of the present application, the concentration of the oligopeptide may be in the range of, but not limited to, about 10 nM to about 100 μM.

In one embodiment of the present application, if the concentration of the oligopeptide is lower than about 0.01 μM or higher than about 100 μM, the effect of the oligopeptide on differentiation into chondrocytes may be lowered. Thus, the concentration of the oligopeptide is preferably in the range of about 0.01 μM to about 100 μM, for example, about 0.01 μM to about 100 μM, about 0.01 μM to about 75 μM, about 0.01 μM to about 50 μM, about 0.01 μM to about 10 μM, about 0.01 μM to about 1 μM, about 0.01 μM to about 0.1 μM, about 0.1 μM to about 100 μM, about 0.1 μM to about 75 μM, about 0.1 μM to about 50 μM, about 0.1 μM to about 10 μM, about 0.1 μM to about 1 μM, about 1 μM to about 100 μM, about 1 μM to about 75 μM, about 1 μM to about 50 μM, about 1 μM to about 10 μM, about 10 μM to about 100 μM, about 10 μM to about 75 μM, about 10 μM to about 50 μM, about 50 μM to about 100 μM, about 50 μM to about 75 μM, or about 75 μM to 100 μM.

In one embodiment of the present application, the stem cells may comprise, but are not limited to, those selected from the group consisting of mesenchymal stem cells, bone marrow-derived stem cells, embryonic stem cells, and combinations thereof.

In one embodiment of the present application, treating the stem cells with the oligopeptide may include, but is not limited to, culturing the stem cells in a medium containing the oligopeptide. The medium may include, but is not limited to, any medium that is generally used for culturing of stem cells. For example, the medium may be, but is not limited to, DMEM, MEM, BME, RPMI 1640, F-10, F-12, DMEM-F12, α-MEM, G-MEM, MSCGM, IMDM, MacCoy's 5A, AmnioMax, AminoMaxII complete Medium, or Chang's Medium MesemCult-XFMedium.

In one embodiment of the present application, the expression of genes or proteins contained in the chondrocytes that differentiated from stem cells may be amplified by treating the stem cells with the oligopeptide. For example, the genes or proteins may include biomarkers. Specifically, the genes or proteins may be selected from the group consisting of collagen type II alpha 1 (COL II), SRY-box 9 (SOX 9), cartilage oligomer matrix protein (COMP), agrican (ACAN), collagen type X alpha 1 (COL X), collagen type I alpha 2 (COL I α2), glyceraldehyde 3-phosphate dehydrogenase (GAPDH), sulfated glycosaminoglycan (sGAG), proteoglycans, and combinations thereof, but are not limited thereto.

In one embodiment of the present application, the stem cells may include, but are not limited to, those selected from the group consisting of mesenchymal stem cells, induced pluripotent stem cells, embryonic stem cells, and combinations thereof.

In one embodiment of the present application, the mesenchymal stem cells may include, but are not limited to, those selected from the group consisting of bone marrow-derived stem cells, adipose-derived stem cells, tonsil-derived stem cells, synovial stem cells, and combinations thereof. For example, the mesenchymal stem cells passaged 2 to 10 times after isolation thereof may be used in the method for differentiation into chondrocytes.

In one embodiment of the present application, the mesenchymal stem cells may be obtained from, but not limited to, bone marrow, tissue, embryo, umbilical cord blood, blood, or body fluid.

In one embodiment of the present application, the method for inducing differentiation of stem cells into chondrocytes may be performed using, but not limited to, a three-dimensional culture method known in the art. The term “three-dimensional culture” refers to a culture method in which cells grow in a cluster so as to form a three-dimensional shape, for example, a sphere or ellipsoid, not a conventional cell monolayer culture method in which cells are cultured so that a cell monolayer is formed by cell growth. Preferably, pellet culture may be used. For example, the pellet culture is effective in maintaining the chondrocyte phenotype, and may provide an extracellular environment similar to that found in the initial cartilage tissue generation by inducing cell-cell adhesion through easy aggregation of cells by centrifugation. In addition, for example, the three-dimensional culture may include culturing the cells or stem cells using a three-dimensional matrix with a hydrogel containing hyaluronic acid or the like.

A third aspect of the present application provides a pharmaceutical composition for treating cartilage injury disease, the pharmaceutical composition containing differentiated chondrocytes obtained by the method according to the second aspect of the present application.

With respect to the pharmaceutical composition according to the third aspect of the present application, detailed description of parts overlapping with those in the first aspect or second aspect of the present application are omitted, but the contents described in the first aspect or second aspect of the present application may be equally applied to the third aspect of the present application.

In one embodiment of the present application, the cartilage injury disease may include, but is not limited to, one selected from the group consisting of arthritis, cartilage injury, cartilage defect, degenerative arthritis, rheumatoid arthritis, fracture, plantar fasciitis, lateral humerus epicondylitis, osteomalacia, and combinations thereof.

In one embodiment of the present application, the pharmaceutical composition may promote, but not limited to, the regeneration of cartilage tissue in the joint by inducing specific differentiation of endogenous stem cells or transplanted therapeutic stem cells into chondrocytes.

In one embodiment of the present application, the pharmaceutical composition may be injected directly into a patient's joint according to a known method, or may be transplanted together with a scaffold after three-dimensional culture, but the present disclosure may not be limited thereto. For example, where the pharmaceutical composition is injected into a patient, the dosage of injection thereof may be adjusted in consideration of several related factors such as the disease to be treated, the severity of the disease, the route of administration, the patient's weight, age and sex.

In one embodiment of the present application, the pharmaceutical composition may further contain a known carrier that is used in the art for injection or transplantation into a patient.

For example, the active ingredient may be suspended or dissolved in a pharmaceutically acceptable carrier according to a conventional method, but the present application may not be limited thereto.

In one embodiment of the present application, the pharmaceutical composition may be used as, but not limited to, oral formulations, such as powders, granules, tablets, suspensions, emulsions and syrups, according to a conventional method.

In one embodiment of the present application, the pharmaceutical composition may be administered orally or parenterally, and the dosage form thereof may vary depending on the method of use.

A fourth aspect of the present application provides a hydrogel for inducing differentiation of stem cells into chondrocytes, the hydrogel containing the composition for inducing differentiation according to the first aspect of the present application.

With respect to the hydrogel for inducing differentiation according to the fourth aspect of the present application, detailed description of parts overlapping with those in the first aspect, second aspect or third aspect of the present application are omitted, but the contents described in the first aspect, second aspect or third aspect of the present application may be equally applied to the fourth aspect of the present application.

In one embodiment of the present application, the hydrogel containing the composition for inducing differentiation may be used together with a scaffold, but the present application may not be limited thereto. For example, the hydrogel containing the composition for inducing differentiation may be applied as a material for tissue engineering together with a scaffold, but the present application may not be limited thereto.

In one embodiment of the present application, the hydrogel containing the composition for inducing differentiation may undergo sol-gel transition by one selected from the group consisting of, but not limited to, chemical reaction, temperature, pH, or UV irradiation, and combinations thereof. For example, the hydrogel containing the composition for inducing differentiation may change from a sol state to a gel state by chemical reaction, temperature, pH or UV irradiation, but the present application may not be limited thereto.

Hereinafter, the present application will be described in more detail with reference to examples, but the present application is not limited by these examples.

### MODE FOR INVENTION

### Examples

Two-Dimensional Culture of Tonsil-Derived Mesenchymal Stem Cells

In this example, tonsil-derived mesenchymal stem cells (TMSCs) recovered from tonsil tissue after tonsillectomy were used as adult stem cells. The stem cell density of tonsil tissue is about 10 to 100 times higher than the stem cell density in bone marrow. In addition, tonsil-derived mesenchymal stem cells proliferate 2 to 3 times faster than bone marrow-derived mesenchymal stem cells. As a first trial to select oligopeptides, tripeptides with aromatic group (phenylalanine, tyrosine, tryptophan) and carboxylic acid (aspartic acid, glutamic acid) were selected because both kartogenin (KGN) and the oxopiperazine derivative 5{1,2} contain the aromatic ring and the carboxylic acid in their structure.

Top three tripeptides exhibiting chondrogenic biomarker expression were screened first, and then the effects thereof on chondrogenic differentiation of tonsil-derived mesenchymal stem cells (TMSCs) were investigated in detail by culturing a 3D pellet for 21 days. Chondrogenic biomarker expression from the cells in the presence of tripeptide or in the absence of tripeptide (negative control) in a serum-free chondrogenic induction medium was investigated. Kartogenin was used as a positive control.

Tonsil-derived mesenchymal stem cells were donated from Ewha Womans University Mokdong Hospital (Seoul, Korea). The cells were isolated from a 6-year-old male donor after tonsillectomy according to NIH guidelines. Tonsil-derived mesenchymal stem cells were two-dimensionally (2D) cultured to passage 5 on plates in a growth medium of high-glucose DMEM (Hyclone, USA) supplemented with 10% (v/v) FBS, 1.0% (v/v) antibiotic/antimitotic solution (Gibco, USA) and 1.0% (v/v) penicillin/streptomycin solution at 37° C. under 5% carbon dioxide. Then, the tonsil-derived mesenchymal stem cells (passage 6) were two-dimensionally (2D) cultured at 37° C. under 5% carbon dioxide. After 24 hours, the growth medium (3.0 mL) supplemented with 10% (v/v) FBS, 1.0% (v/v) antibiotic/antimitotic solution (Gibco, USA) and 1.0% (v/v) penicillin/streptomycin solution was replaced by the same growth medium free of FBS, and each tripeptide or kartogenin was added to the solution. Control (negative) experiments were performed in the absence of each tripeptide or kartogenin. Kartogenin-containing protocol is used as a positive control experiment. The medium was replaced every three days. The same numbers of tonsil-derived mesenchymal stem cells (about 2.5×10⁵ cells/well) were used for each system. Each experiment was triplicate (n=3).

Proliferation of Tonsil-Derived Mesenchymal Stem Cells

A cell counting kit-8 (CCK-8) solution was prepared in high-glucose DMEM (Hyclone, USA) supplemented with 1.0% (v/v) penicillin/streptomycin solution. The solution (0.5 mL) replaced the medium of each cell culture well in which tonsil-derived mesenchymal stem cells were incubated for 3 days in the growth medium supplemented with each tripeptide (10 μM) or kartogenin, 1.0% (v/v) antibiotic/antimitotic solution (Gibco, USA), and 1.0% (v/v) penicillin/streptomycin solution. After incubation for 3 hours at 37° C. under the 5% carbon dioxide, the absorbance of the sample was measured at 450 nm relative to 655 nm by using a microplate reader (iMark™, Bio-Rad, USA). The absorbance was converted to cell viability relative to the control group which does not contain each tripeptide or kartogenin.

Live/Dead Test of Cultured Stem Cells

Tonsil-derived mesenchymal stem cells were cultured in the DMEM supplemented with each tripeptide (10 μM) or kartogenin, 1.0% (v/v) antibiotic/antimitotic solution (Gibco, USA) and 1.0% (v/v) penicillin/streptomycin solution for 7 days. The media were replaced by phosphate buffered saline (PBS) containing ethidium homodimer-1 (4.0 μM) and calcein acetoxy methyl ester (2.0 μM), and the cells were incubated for 15 minutes. Viability of tonsil-derived mesenchymal stem cells was assayed by the Live/Dead kit (Molecular Probes, Life Technologies, USA) using an Olympus IX71 fluorescence microscope and the Olympus DP2-BSW software.

Three-Dimensional Pellet Culture of Stem Cells

Tonsil-derived mesenchymal stem cells (passage 6) were enzymatically detached by trypsin treatment and counted by a hemocytometer. The tube was centrifuged in 500-g relative centrifugal force (RCF) for 10 minutes and incubated at 37° C. under 5% carbon dioxide for 24 hours. A cell pellet consisting of about 3.0×10⁵ cells was formed in a conical polypropylene tube (15 mL). The FBS-containing growth medium was replaced by medium A containing a high-glucose DMEM supplemented with 1.0% (v/v) antibiotic/antimitotic solution, 1.0% (v/v) penicillin/streptomycin solution, 50 μg/mL ascorbate-2-phosphate, 40 μg/mL L-proline, and 1% ITS+Premix (final concentration: 10 μg/mL bovine insulin, 5.5 μg/mL transferrin, 5 μg/mL sodium selenite, 4.7 μg/mL linoleic acid, and 0.5 mg/mL BSA), and 100 nM dexamethasone. Pellets were cultured at 37° C. under 5% carbon dioxide for 21 days under the following three different conditions: 1) medium A only, 2) medium A supplemented with kartogenin, or 3) medium A supplemented with each tripeptides. Each medium was replaced every three days. Triplicate experiments were carried out for each system.

RNA Extraction and Real-Time RT-PCR

Total RNA was extracted from the cells by using a TRIZOL™ reagent (Invitrogen, USA). The mRNA concentration was assayed by Nano Drop 2000 spectrophotometer (Thermo Scientific, USA). cDNA was synthesized by ReverTra Ace® qPCR RT kit (Toyobo, Japan). mRNA expression levels were analyzed by real-time RT-PCR (CFX96™, Bio-Rad, USA) using IQ™ SYBR® Green Supermix (Bio-Rad, USA). The expression levels of target genes were calculated as 2^{-ΔΔCt} by the following calculation equation, and the primer sequences are shown in Table 1 below.

ΔΔCt=(gene A−GAPDH)−(gene A−GAPDH)_{control}

TABLE 1

| Genes | Primer sequences |
|---|---|
| COL II | F: 5′-CACTGGCAGTGGCGAGGTCAG-3′ |
| | R: 5′-CACTGGCAGTGGCGAGGTCAG-3′ |
| SOX 9 | F: 5′-GCAGCGAAATCAACGAGAAAC-3′ |
| | R: 5′-TCCAAACAGGCAGAGAGATTTAG-3′ |
| COMP | F: 5′-AGCAGATGGAGCAAACGTATTG-3′ |
| | R: 5′-ACAGCCTTGAGTTGGATGCC-3′ |
| ACAN | F: 5′-CAGAGCAAGACTCTGTCTCAAA |
| | R: 5′-GGGACAAGAGCCTCATCAAA-3′ |
| COL X | F: 5′-CAAGGCACCATCTCCAGGAA-3′ |
| | R: 5′-AAGGGTATTTGTGGCAGCATATT-3′ |
| COL I α2 | F: 5′-GGACACAATGGATTGCAAGG-3′ |
| | R: 5′-TAACCACTGCTCCACTCTGG-3′ |
| GAPDH | F: 5′-CTCCTCACAGTTGCCATGTA-3′ |
| | R: 5′-GTTGAG5CACAGGGTACTTTATTG-3′ |

In Table 1 above, COL II, SOX 9, COMP, ACAN, COL X, COL I α2, and GAPDH indicate collagen type II alpha 1, SRY-box 9, cartilage oligomeric matrix protein, aggrecan, collagen type X alpha 1, collagen type I alpha 2, and glyceraldehyde 3-phosphate dehydrogenase, respectively. F and R indicate forward and reverse primers, respectively.

Histological Staining

The stem cell pellets were fixed in formalin aqueous solution (10% v/v) and embedded in paraffin. Then, these pellets were sectioned to a thickness of 7 μm by using a microtome. Then, the sectioned cell pellet samples were deparaffinized by xylene, and analyzed by immunofluorescence using antibody, alcian blue staining, and safranin O staining to visualize COL II, sGAG and proteoglycan, respectively.

For immunofluorescent assay, the sectioned pellet samples were permeabilized by triton (0.1% v/v in PBS) and treated with BSA (1% wt/v in PBS, Sigma, USA). COL II was assayed using goat anti-mouse H&L Alexa Fluor® 594 (Abcam, USA) and goat anti-rabbit IgG H&L Alexa Fluor® 594 (Abcam, USA). The nucleus and actin were stained with DAPI (Molecular probes, USA) and phalloidin (Abcam, USA), respectively.

For alcian blue staining, the sectioned samples were treated with alcian blue solution (1% wt./v in acetic acid aqueous solution (3.0% v/v), Sigma Aldrich, USA) and Nuclear Fast Red aqueous solution (0.1% wt/v, Sigma Aldrich, USA).

For safranin O staining, the sectioned samples were treated with a Fast Green FCF aqueous solution (0.001% wt./v, Sigma Aldrich, USA) and safranin O aqueous solution (0.1% wt/v Acros Organics, USA) according to the manufacturer's protocol.

Among various combination of tripeptides, alanine-alanine-glutamic acid (AAE), phenylalanine-phenylalanine-glutamic acid (FFE), isoleucine-phenylalanine-glutamic acid (IFE), leucine-phenylalanine-glutamic acid (LFE), methionine-phenylalanine-glutamic acid (MFE), valine-phenylalanine-glutamic acid (VFE), tryptophan-phenylalanine-glutamic acid (WFE), tyrosine-phenylalanine-glutamic acid (YFE), phenylalanine-arginine-aspartic acid (FRD), phenylalanine-leucine-glutamic acid (FLE), tryptophan-leucine-glutamic acid (WLE), tyrosine-isoleucine-aspartic acid (YID), tyrosine-tyrosine-aspartic acid (YYD), and tyrosine-tyrosine-glutamic acid (YYE) were selected as candidate promoters for chondrogenic differentiation of tonsil-derived mesenchymal stem cells. Targeting the effectiveness of each tripeptide similar to those of KGN and the oxopiperazine derivative 5{1,2}, tonsil-derived mesenchymal stem cells were cultured in a serum-free growth medium of DMEM in the presence (10 μM) or absence of each tripeptide. The cell density did not significantly change and the cells remained alive, as exhibited in green by Live/Dead images and CCK-8 assay, over the 3 days of incubation. In the first round of screening, chondrogenic biomarker expression was investigated at the mRNA level by real-time reverse transcription polymerase chain reaction (RT-PCR) for collagen type II (COL II), SOX 9, and cartilage oligomer matrix protein (COMP) after 7 days of incubation. The biomarker expression was enhanced in the presence of WFE, WLE, and YYE. YYE acted as a good promoter comparable to kartogenin in the chondrogenic biomarker expression. In particular, the structural uniqueness of YYE can be compared with those of FFE and YFE. The hydroxyl group of Y in the first and second amino acids will be important for becoming a promoter of chondrogenic differentiation of tonsil-derived mesenchymal stem cells.

Based on the above prescreening information, WFE, WLE and YYE were investigated as a promoter of chondrogenic differentiation in detail by using 3D pellet culture in chondrogenic medium. The medium was supplemented with each tripeptide (10 μM), and tonsil-derived mesenchymal stem cells (size: ˜ mm) were prepared. Chondrogenic medium without tripeptide was used as control experiment. Medium supplemented with kartogenin (target molecule) instead of tripeptide was used for the purpose of comparing chondrogenic differentiation of tonsil-derived mesenchymal stem cells. Compared with day zero, the cell density increased 1.4 to 1.8-fold and 2.4 to 2.6-fold, respectively during the 3D culture periods of 14 and 21 days, respectively.

Chondrogenic biomarker expression for COL II, COMP and ACAN was analyzed at the mRNA level by RT-PCR. COMP interacts with extracellular proteins such as collagen, fibronectin, TGF-β, etc. and is more abundant in healthy cartilage than cartilage with osteoarthritis. All of these chondrogenic mRNAs of COL II, COMP, and ACAN were highly expressed by the tripeptides WFE, WLE and YYE selected in the second round. In particular, YYE increased the expression levels of COL II, COMP and ACAN by 1.7 to 2.8-fold, but decreased the expression levels of COL X and COL I α2 to less than half of the control system, suggesting that YYE was effective in inducing chondrogenic differentiation of the stem cells. COL X is a biomarker related to the hypertrophy of the cells which is an intermediate state leading to osteogenic differentiation, and COL I α2 is a biomarker related to osteogenic differentiation of stem cells. Osteogenic differentiation of stem cells should be interrupted at the chondrogenic differentiation stage of the stem cells, and controlling this osteogenic differentiation is a critical issue for osteoarthritis treatment. In this regard, the fact that YYE specifically induces chondrogenic differentiation and suppresses osteogenic differentiation of tonsil-derived mesenchymal stem cells suggests that YYE down-regulates osteogenic differentiation of tonsil-derived mesenchymal stem cells. COL II accounts for 80% to 90% of collagen in articular cartilage and is most widely used as a biomarker for chondrogenic differentiation of mesenchymal stem cells. In addition, COL II was analyzed at protein level by immunofluorescence assay, where the antibody of COL II was stained in red. The nucleus and actin of cells were stained in blue and green by 4′,6-diamidino-2-phenylindole (DAPI) and phalloidin, respectively. The YYE- and kartogenin-treated systems were distinguished for their red staining by COL II, compared with the control system and the WFE- or WLE-treated system.

Sulfated glycosaminoglycan (sGAG) and proteoglycan are typical biomarkers of the chondrogenic differentiation of mesenchymal stem cells. The 3D cultured pellet was sliced and stained by alcian blue and safranin O which stain sGAG and proteoglycan in blue and red, respectively. The tonsil-derived mesenchymal stem cells cultured in the presence of tyrosine-tyrosine-glutamic acid upregulated the chondrogenic biomarkers sGAG and proteoglycan, similar to kartogenin.

In the present application, it was confirmed that a series of tripeptides are effective in inducing the differentiation of tonsil-derived mesenchymal stem cells into chondrocytes. In 2D culture, tryptophan-phenylalanine-glutamic acid (Trp-Phe-Glu, WFE), tryptophan-leucine-glutamic acid (Trp-Leu-Glu, WLE), tyrosine-tyrosine-glutamic acid (Tyr-Tyr-Glu, YYE) were selected based on the chondrogenic biomarker expression. Tonsil-derived mesenchymal stem cells were made into three-dimensional pellets and cultured for 21 days in chondrogenic medium in the presence of each tripeptide as a promoter of chondrogenic differentiation of tonsil-derived mesenchymal stem cells. Not only alcian blue straining and safranin O staining of the cell pellets, but also expression of the biomarkers COL II, COMP, ACAN, COL X and COL I α2, demonstrated that, among the oligopeptides, an oligopeptide containing an amino acid sequence of tyrosine-tyrosine-glutamic acid is an excellent compound for enhancing chondrogenic differentiation of tonsil-derived mesenchymal stem cells while suppressing osteogenic differentiation of the stem cells.

The above description of the present application is exemplary, and those of ordinary skill in the art to which the present application pertains will appreciate that the present application can be easily modified into other specific forms without departing from the technical spirit or essential characteristics of the present application. Therefore, it should be understood that the embodiments described above are exemplary in all aspects and are not restrictive. For example, each component described to be of a single type can be implemented in a distributed manner. Likewise, components described to be distributed can be implemented in a combined manner.

It shall be understood that the scope of the present application is defined by the following claims rather than by the above detailed description, and all modifications and variations conceived from the meaning and scope of the claims and equivalents thereto are included within the scope of the present application.

### INDUSTRIAL APPLICABILITY

The material of the present application may be used to induce differentiation of stem cells into chondrocytes and used as a material for cartilage disease treatment or tissue engineering.

1. A composition for inducing differentiation of stem cells into chondrocytes, the composition containing an oligopeptide consisting of 2 to 10 amino acids.

2. The composition of claim 1, wherein the oligopeptide comprises amino acids selected from the group consisting of glycine, alanine, valine, leucine, isoleucine, phenylalanine, proline, tyrosine, tryptophan, cystine, methionine, serine, threonine, lysine, arginine, histidine, aspartic acid, glutamic acid, asparagine, glutamine, and combinations thereof.

3. The composition of claim 2, wherein the oligopeptide consists of an amino acid sequence of alanine-alanine-glutamic acid, phenylalanine-phenylalanine-glutamic acid, isoleucine-phenylalanine-glutamic acid, leucine-phenylalanine-glutamic acid, methionine-phenylalanine-glutamic acid, valine-phenylalanine-glutamic acid, tryptophan-phenylalanine-glutamic acid, tyrosine-phenylalanine-glutamic acid, phenylalanine-arginine-aspartic acid, phenylalanine-leucine-glutamic acid, tryptophan-leucine-glutamic acid, tyrosine-isoleucine-aspartic acid, tyrosine-tyrosine-aspartic acid, or tyrosine-tyrosine-glutamic acid.

4. The composition of claim 1, wherein a concentration of the oligopeptide is in the range of 10 nM to 100 μM.

5. A method for inducing differentiation of stem cells into chondrocytes, the method comprising inducing differentiation into chondrocytes by treating the stem cells with an oligopeptide consisting of 2 to 10 amino acids.

6. The method of claim 5, wherein the oligopeptide comprises amino acids selected from the group consisting of glycine, alanine, valine, leucine, isoleucine, phenylalanine, proline, tyrosine, tryptophan, cystine, methionine, serine, threonine, lysine, arginine, histidine, aspartic acid, glutamic acid, asparagine, glutamine, and combinations thereof.

7. The method of claim 6, wherein the oligopeptide consists of an amino acid sequence of alanine-alanine-glutamic acid, phenylalanine-phenylalanine-glutamic acid, isoleucine-phenylalanine-glutamic acid, leucine-phenylalanine-glutamic acid, methionine-phenylalanine-glutamic acid, valine-phenylalanine-glutamic acid, tryptophan-phenylalanine-glutamic acid, tyrosine-phenylalanine-glutamic acid, phenylalanine-arginine-aspartic acid, phenylalanine-leucine-glutamic acid, tryptophan-leucine-glutamic acid, tyrosine-isoleucine-aspartic acid, tyrosine-tyrosine-aspartic acid, or tyrosine-tyrosine-glutamic acid.

8. The method of claim 5, wherein treating the stem cells with the oligopeptide comprises culturing the stem cells in a medium containing the oligopeptide.

9. The method of claim 8, wherein a concentration of the oligopeptide in the medium is in the range of 10 nM to 100 μM.

10. The method of claim 5, wherein the stem cells comprise those from the group consisting of mesenchymal stem cells, induced pluripotent stem cells, embryonic stem cells, and combinations thereof.

11. A pharmaceutical composition for treating cartilage injury disease, the pharmaceutical composition containing differentiated chondrocytes obtained by the method according to claim 5.

12. The pharmaceutical composition of claim 11, wherein the cartilage injury disease includes one selected from the group consisting of arthritis, cartilage injury, cartilage defect, degenerative arthritis, rheumatoid arthritis, fracture, plantar fasciitis, lateral humerus epicondylitis, osteomalacia, and combinations thereof.

13. A hydrogel for inducing differentiation of stem cells into chondrocytes, the hydrogel containing the composition for inducing differentiation according to claim 1.

14. The hydrogel of claim 13, containing a biodegradable polymer selected from the group consisting of hyaluronic acid, polypeptide, polyester, polycarbonate, and combinations thereof.
